(19) United States
(12) Patent Application Publication
(10) Pub. No.: US 2012/0156304 A1
(43) Pub. Date: Jun. 21, 2012

(54) BRANCHED POLYOL POLYESTERS, BLENDS, AND PHARMACEUTICAL FORMULATIONS COMPRISING SAME

(76) Inventor: Thomas Tice, Indian Springs, AL (US)

(21) Appl. No.: 13/327,106

(22) Filed: Dec. 15, 2011

Related U.S. Application Data
(60) Provisional application No. 61/423,140, filed on Dec. 15, 2010.

(51) Int. Cl. A61K 9/14; A61K 38/12; C08G 63/91

(52) U.S. Cl. 424/497; 525/54.2; 514/21.1

(57) ABSTRACT

Disclosed herein are branched polyol polyesters, which are useful in pharmaceutical formulations. Also disclosed are blends, microparticles, and other formulations, comprising the branched polyol polyesters.

This Application claims the benefit of U.S. Provisional Application No. 61/423,140, filed on Dec. 15, 2010 which is incorporated herein by reference in its entirety.

### BACKGROUND

Bioactive agents are often delivered to a subject through a formulation such as a microparticle, which comprises a biodegradable polymer such as a biodegradable polymer that can be synthesized from a cyclic lactone or carbonate like lactide, glycolide, caprolactone, dioxanone, or trimethylene carbonate to make poly, or a copolymer thereof, such as a terpolymer. Certain bioactive agents have a tendency to release quickly from such formulations (i.e., burst). Also, burst can occur when a formulation has a high loading of a bioactive agent; the high loading is needed because the bioactive agent may have a low potency or a large amount of the bioactive agent is needed for the desired dose to be available for a long duration. Accordingly, a need exists for improved biodegradable formulations that address the issue of the burst of the bioactive agent from the formulation. These needs and other needs are satisfied by the present invention.

### SUMMARY

Disclosed herein are branched polyesters, in particular branched polyesters of a polyol, having a molecular weight of less than 20,000 Daltons, or, alternatively, greater than 200,000 Daltons; wherein the polyester comprises one or more residues of lactide, glycolide, caprolactone, dioxanone, or trimethylene carbonate.

Also disclosed are blends comprising a first branched polyester of a polyol having a molecular weight less than 20,000 Daltons together with a second branched polyester of a polyol having a molecular weight greater than 200,000 Daltons. Such blends can also comprise other components, such as a linear polyester.

Also disclosed are blends comprising a linear polyester and a branched polyester of a polyol. For example, a high-molecular-weight linear polyester can be blended together with lower-molecular-weight branched polyester of a polyol, wherein the branched polyester has a molecular weight less than 20,000 Daltons. Alternatively, a linear polyester can be blended together with a high molecular weight branched polyester of a polyol, wherein the branched polyester has a molecular weight greater than 200,000 Daltons.

Also disclosed are methods of modulating burst of a bioactive agent from a formulation comprising a linear polyester of a polyol comprising blending a branched polyester of a polyol with the linear polyester.

Also disclosed are microparticles, implants, films, fibers, liquid polymers, and other formulations comprising the disclosed polymers and blends. Also disclosed are microparticles comprising a matrix formed from a linear polyester and a branched polyester and a bioactive agent encapsulated therein wherein the branched polyester reduces the burst of the bioactive agent.

### DETAILED DESCRIPTION

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

The singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The term “microparticle” is used herein to refer generally to a variety of structures having sizes from about 10 nm to 2000 microns (2 millimeters) and includes microcapsule, microsphere, nanoparticle, nanocapsule, nanosphere as well as particles, in general, that are less than about 2000 microns (2 millimeters). In one aspect, the bioactive agent is encapsulated in the microparticle.

The term “biocompatible” refers to a substance and its degradation products that are substantially non-toxic to a subject.

“Biodegradable” is generally referred to herein as a material that will erode to soluble species or that will degrade under physiologic conditions to smaller units or chemical species that are, themselves, non-toxic (biocompatible) to the subject and capable of being metabolized, eliminated, or excreted by the subject. A “biodegradable” substance may also be referred to as bioresorbable or simply, resorbable.

Disclosed herein, in one aspect, are branched polyesters, in particular branched polyesters of a polyol, having a molecular weight of less than 20,000 Daltons, or, alternatively, greater than 200,000 Daltons; wherein the polyester comprises one or more residues of lactide, glycolide, caprolactone, dioxanone, or trimethylene carbonate.

As used herein calculations of molecular weight and references to a particular weight refer to weight average molecular weight (Mw). Molecular weights of the linear and branched polyesters disclosed herein can be determined using conventional methods, such as gel permeation chromatography (GPC) using a polystyrene as standard (Mw). The molecular weights (Mw) of the branched polyesters are either less than 20,000 Daltons, or, alternatively, greater than 200,000 Daltons. The molecular weights (Mw) of linear polymers can be up to 500,000 Daltons. It is understood that where a particular molecular weight or molecular weight range for a given branched or linear polyester is disclosed, said molecular weight or molecular weight range is contemplated for each and every aspect of the compositions and methods disclosed herein including the blended compositions.

In one aspect disclosed herein are branched polyesters of a polyol, having a molecular weight of less than 19990, 19980, 19970, 19960, 19950, 19900, 19800, 19700, 19600, 19500, 19400, 19300, 19200, 19000, 18500, 18000, 17500, 17000, 16500, 16000, 15500, 15000, 14500, 14000, 13500, 13000, 12500, 12000, 11500, 11000, 10500, 10000, 9000, 8000, 7000, 6000, 5000, 4000, 3000, 2000, or 1000 Daltons. For example, in one aspect the dislcosed branched polyesters of a polyol can have a molecular weight of 1000, 1500, 2000, 2500, 3,000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 185000, 19000, 19500, 19600, 19700, 19800, 19900, 19950, 19960, 19970, 19980, 19990, 19995 or 19999 Daltons. In yet another aspect, the disclosed branched polyesters of a polyol can have a molecular weight from 1000 to 20000; from 1000 to 19900; from 1000 to 19500, from 1000 to 19000; from 1000 to 18000; from 1000 to 17000; from 1000 to 16000; from 1000 to 15000; from 1000 to 10000; from 1000 to 5000; from 5000 to 20000; from 5000 to 19900; from 5000 to 195000; from 5000 to 19000; from 5000 to 18000; from 5000 to 17000; from 5000 to 16000; from 5000 to 15000; from 5000 to 10000; from 10000 to 20000; from 10000 to 19900; from 10000 to 19500; from 10000 to 19000; from 10000 to 18000; from 10000 to 17000; from 10000 to 16000; from 10000 to 15000; from 15000 to 20000; from 15000 to 19900; from 15000 to 19500; from 15000 to 19000; from 15000 to 18000; from 15000 to 17000; or 15000 to 16000 Daltons.

In another aspect, disclosed herien are branched polyesters of a polyol, having a molecular weight of greater than 200,000 Daltons. In another aspect disclosed herien are polyesters of a polyol having a molecular weight greater than 200100, 200200, 200300, 200400, 200500, 200600, 200700, 200800, 200900, 201000, 202000, 203000, 204000, 205000, 210000, 215000, 220000, 225000, 230000, 235000, 240000, 245000, 250000, 275000, 300000, 350000, 400000, 450000, or 500000 Daltons. For example, the branched polyester of a polyol can have a molecular weight of 200100, 200200, 200300, 200400, 200500, 200600, 200700, 200800, 200900, 201000, 202000, 203000, 204000, 205000, 210000, 215000, 220000, 225000, 230000, 235000, 240000, 245000, 250000, 275000, 300000, 350000, 400000, 450000, or 500000 Daltons. In yet another aspect, the branched polyester of a polyol can have a molecular weight from 200,000 to 500,000; from 200,000 to 450,000; from 200,000 to 400,000; from 200,000 to 350,000; from 200,000 to 300,000; from 200,000 to 250,000; from 210,000 to 500,000; from 210,000 to 450,000; from 210,000 to 400,000; from 210,000 to 350,000; from 210,000 to 300,000; from 210,000 to 250,000; from 220,000 to 500,000; from 220,000 to 450,000; from 220,000 to 400,000; from 220,000 to 350,000; from 220,000 to 300,000; from 220,000 to 250,000; from 230,000 to 500,000; from 230,000 to 450,000; from 230,000 to 400,000; from 230,000 to 350,000; from 230,000 to 300,000; from 230,000 to 250,000; from 240,000 to 500,000; from 240,000 to 450,000; from 240,000 to 400,000; from 240,000 to 350,000; from 240,000 to 300,000; from 240,000 to 250,000; from 250,000 to 500,000; from 250,000 to 450,000; from 250,000 to 400,000; from 250,000 to 350,000; from 250,000 to 300,000; from 300,000 to 500,000; from 300,000 to 450,000; from 300,000 to 400,000; from 300,000 to 350,000; from 350,000 to 500,000; from 350,000 to 450,000; from 350,000 to 400,000; from 400,000 to 500,000; 400,000 to 450,000; or from 450,000 to 500,000 Daltons.

The term “branched polyester,” or “branched polyester of a polyol,” refers to a polyester having a core (which will typically be the initiator used for polymerization) having at least two branches, for example 3, 4, 5, 6, 7, 8, 9, or 10, radiating therefrom. It is understood and herein contemplated that the branched polyester an be any branched polymer type including but not limited to comb polymer, star polymer, brushed polymer, graft polymer, or other polymer network.

As used herein, the term “star polymer” or “star polyester” refers to a branched polymer molecule in which a single branch point gives rise to multiple linear chains or arms. If the arms are identical the star polymer molecule is said to be regular. If adjacent arms are composed of different repeating subunits, the star polymer molecule is said to be variegated. In one aspect, disclosed herien are star-type branched polyesters, in particular branched polyesters of a polyol, having a molecular weight of less than 20,000 Daltons, or, alternatively, greater than 200,000 Daltons; wherein the polyester comprises one or more residues of lactide, glycolide, caprolactone, dioxanone, or trimethylene carbonate.

Also disclosed herein are linear polyesters comprising a linear polyester of a polyol having a molecular weight up to 500,000 Daltons, wherein the polyester comprises one or more residues of lactide, glycolide, caprolactone, dioxanone, or trimethylene carbonate. As used herein, the term “linear” polymer or “linear” polyester refers to a polymer or polyester that is not branched, an example of which is a polyester initiated by a monofunctional initiator, such that a single polymer chain grows. In one aspect disclosed herein are linear polyesters of a polyol having a molecular weight up to 500,000 Daltons, wherein the polyester comprises one or more residues of lactide, glycolide, caprolactone, dioxanone, or trimethylene carbonate. In one aspect disclosed herein are branched polyesters of a polyol, having a molecular weight of 1000, 1500, 2000, 2500, 3,000, 3500, 4000, 4500, 5000, 6000, 7000, 8000, 9000, 9500, 10000, 11000, 12000, 13000, 14000, 15000, 16000, 17000, 18000, 19000, 20000, 21000, 22000, 23,000, 24000, 25000, 30000, 35000, 40000, 45000, 50000, 60000, 70000, 80000, 90000, 10000, 125000, 15000, 175000, 200000, 250000, 300000, 350000, 400000, 450000, or 500000 Daltons. For example, in one aspect the linear polyester can have a molecular weight from 1,000 to 500,000; 1,000, to 300,000; 1,000 to 100,000; 1,000 to 50,000; 1,000 to 25,000; 1,000 to 20,000; 1,000 to 10,000; 10,000 to 50,000; 10,000 to 300,000; 10,000 to 100,000; 10,000 to 50,000; 50,000 to 500,000; 50,000 to 300000; 50,000 to 200,000; 50,000 to 100,000; 100,000 to 500,000; 100,000 to 300,000; 100,000 to 200,000; 200,000 to 500,000; 200,000 to 300,000 or 300,000 to 500,000 Daltons.

The branched and linear polyesters disclosed herein are useful in formulations comprising bioactive agents. A “bioactive agent” refers to an agent that has biological activity. The biological agent can be used to treat, diagnose, cure, mitigate, prevent (i.e., prophylactically), ameliorate, modulate, or have an otherwise favorable effect on a disease, disorder, infection, and the like. A “releasable bioactive agent” is one that can be released from a disclosed formulation. Bioactive agents also include those substances which affect the structure or function of a subject, or a pro-drug, which becomes bioactive or more bioactive after it has been placed in a predetermined physiological environment. In particular, the branched polyesters are useful in decreasing burst release of a bioactive agent from a formulation, such as a microparticle, when present in a biological environment, such as within a subject. Generally, the disclosed branched polyesters are polyesters of a polyol. Thus, for example, disclosed herein are star type branched polyesters of a polyol having a molecular weight of less than 20,000 Daltons, or, alternatively, greater than 200,000 Daltons.

In one aspect, the disclosed branched and linear polyester comprises one or more residues of lactide, glycolide, caprolactone, dioxanone, or trimethylene carbonate. In one aspect, the branched polyester is a poly(lactide-co-glycolide) ester of the polyol. The term “polyester of a polyol” refers to a polyol having at least two hydroxyl groups that are esterified with a polymer chain, such as poly(lactide-co-glycolide). For example, disclosed herein are branched polyesters, such as, for example, a star type branched polyester, of a polyol having a molecular weight of less than 20,000 Daltons, or, alternatively, greater than 200,000 Daltons; wherein the polyester comprises one or more residues of lactide, glycolide, caprolactone, dioxanone, or trimethylene carbonate. Also disclosed, for example are linear polyesters comprising a linear polyester of a polyol having a molecular weight less than 500,000 Daltons, wherein the polyester comprises one or more residues of lactide, glycolide, caprolactone, dioxanone, or trimethylene carbonate.

The branched and linear polyesters are prepared by initiating the polymerization of a cyclic ester, such as lactide, glycolide, caprolactone, dioxanone, or trimethylene carbonate, using the polyol. The polyol has more than one hydroxyl group, and thus polymerization is initiated at each hydroxyl, creating a branched or star polymer.

The polyol used to initiate the polymerization of the cyclic ester contains at least 2 hydroxyl groups, for example from 2 to 30 hydroxyl groups, or from 3 to 30 hydroxyl groups. Suitable polyols include, without limitation, mannitol, pentaerythritol, sorbitol, ribitol, and xylitol.

Other suitable polyols have a cyclic structure and contain from 4 to 30 hydroxyl groups. The cyclic polyols can contain one or more saccharide units with at least 3 hydroxyl groups per unit. Examples of such polyols are those with a fructose structure, such as fructose, glucose, or a polymer thereof. A polyol can contain multiple fructose or glucose units connected together, such as beta-cyclodextrin, which has multiple glucose units connected through the 1, 4 positions.

The branched polyesters can be prepared by reacting a suitable cyclic monomer in the presence of the polyol and optionally in the presence of a polymerization catalyst. An exemplary polymerization catalyst is Sn-octoate. The reaction components can be mixed together with the catalyst and reacted at an elevated temperature.

When a solvent is used during the reaction, such as toluene, the components can be reacted at the reflux temperature of the solvent. Without a solvent, the reaction temperature can be higher, e.g., if glucose is used as the polyol, up to about 170° C., and if beta-cyclodextrin is used, up to 180° C. The formed polyol polyester can be purified and isolated in a conventional manner.

The molecular weights of the polyol polyesters are dependent on the weight ratio of the components in the reaction (e g., the amount of initiator, and on the reaction conditions, e.g., the reaction temperature). More initiator can lead to shorter polymer chains and thus to lower molecular weight polyol polyesters. The isolation and purification can influence the molecular weight of the purified polyol polyesterester. Changing the isolation and purification conditions can lead to a change of the molecular weight. Because the polyol polyester can exist generally as a mixture of molecules with chains of a different length, the composition of this mixture can be influenced by isolation and purification methods, such as extraction, precipitation, filtration, and isolation. The molecular weight of the purified polymer can be increased by removing low-molecular-weight polyester, e.g., by a suitable precipitation of the polymer, e.g., in methanol, or by membrane filtration.

Certain bioactive agents have a tendency to be released quickly from microparticle formulations (i.e., burst). In particular, this problem can arise where a formulation has a high loading of a bioactive agent. As used herein, “burst” refers to the unintentional release of drug. Typically burst refers to the release of drug or other bioactive agent faster than desired. Burst can occur over minutes, hours, or days. Thus not only is the bioactive agent wasted, there is a possibility that the formulation will have an undesired effect, toxicity, local/site reaction. Burst is amount of drug release early verses % of total dose. Burst can refer to initial drug plasma level verses steady state level. Thus, in one aspect, the disclosed compositions are designed to avoid burst.

The blends can comprise a first branched polyester having a molecular weight of less than 20,000 Daltons together with a second branched polyester having a molecular weight of greater than 200,000 Daltons. Such blends can also comprise additional components, such as the linear polyesters discussed above. For example, in one aspect disclosed herein are blends of branched polyesters of a polyol, wherein the first branched polyester has a molecular weight of less than 19990, 19980, 19970, 19960, 19950, 19900, 19800, 19700, 19600, 19500, 19400, 19300, 19200, 19000, 18500, 18000, 17500, 17000, 16500, 16000, 15500, 15000, 14500, 14000, 13500, 13000, 12500, 12000, 11500, 11000, 10500, 10000, 9000, 8000, 7000, 6000, or 5000 Daltons and the second branched polyester of has a molecular weight greater than 200100, 200200, 200300, 200400, 200500, 200600, 200700, 200800, 200900, 201000, 202000, 203000, 204000, 205000, 210000, 215000, 220000, 225000, 230000, 235000, 240000, 245000, 250000, 275000, 300000, 350000, 400000, 450,000, or 500,000 Daltons. In another aspect are branched polyester blends wherein the first branched polyester has a molecular weight from 1000 to 20000; from 1000 to 19900; from 1000 to 19500, from 1000 to 19000; from 1000 to 18000; from 1000 to 17000; from 1000 to 16000; from 1000 to 15000; from 1000 to 10000; from 1000 to 5000; from 5000 to 20000; from 5000 to 19900; from 5000 to 195000; from 5000 to 19000; from 5000 to 18000; from 5000 to 17000; from 5000 to 16000; from 5000 to 15000; from 5000 to 10000; from 10000 to 20000; from 10000 to 19900; from 10000 to 19500; from 10000 to 19000; from 10000 to 18000; from 10000 to 17000; from 10000 to 16000; from 10000 to 15000; from 15000 to 20000; from 15000 to 19900; from 15000 to 19500; from 15000 to 19000; from 15000 to 18000; from 15000 to 17000; or 15000 to 16000 Daltons and the second branched polyester has a molecular weight from 200,000 to 500,000; from 200,000 to 450,000; from 200,000 to 400,000; from 200,000 to 350,000; from 200,000 to 300,000; from 200,000 to 250,000; from 210,000 to 500,000; from 210,000 to 450,000; from 210,000 to 400,000; from 210,000 to 350,000; from 210,000 to 300,000; from 210,000 to 250,000; from 220,000 to 500,000; from 220,000 to 450,000; from 220,000 to 400,000; from 220,000 to 350,000; from 220,000 to 300,000; from 220,000 to 250,000; from 230,000 to 500,000; from 230,000 to 450,000; from 230,000 to 400,000; from 230,000 to 350,000; from 230,000 to 300,000; from 230,000 to 250,000; from 240,000 to 500,000; from 240,000 to 450,000; from 240,000 to 400,000; from 240,000 to 350,000; from 240,000 to 300,000; from 240,000 to 250,000; from 250,000 to 500,000; from 250,000 to 450,000; from 250,000 to 400,000; from 250,000 to 350,000; from 250,000 to 300,000; from 300,000 to 500,000; from 300,000 to 450,000; from 300,000 to 400,000; from 300,000 to 350,000; from 350,000 to 500,000; from 350,000 to 450,000; from 350,000 to 400,000; from 400,000 to 500,000; 400,000 to 450,000; or from 450,000 to 500,000 Daltons. Examples of blends of a first and second branched polyester can include any combination of the branched polyesters disclosed herein having a molecular weight less than 20,000 Daltons and branched polyesters having a molecular weight more than 200,000 Daltons including but not limited to blends wherein the first branched polyester has a molecular weight of 19900 Daltons and the second branched polyester has a molecular weight of 210,000 Daltons; blends wherein the first branched polyester has a molecular weight of 19800 Daltons and the second branched polyester has a molecular weight of 220,000 Daltons blends wherein the first branched polyester has a molecular weight of 19700 Daltons and the second branched polyester has a molecular weight of 230,000 Daltons blends wherein the first branched polyester has a molecular weight of 19600 Daltons and the second branched polyester has a molecular weight of 240,000 Daltons blends wherein the first branched polyester has a molecular weight of 19500 Daltons and the second branched polyester has a molecular weight of 250,000 Daltons; blends wherein the first branched polyester has a molecular weight of 18000 Daltons and the second branched polyester has a molecular weight of 300,000 Daltons; blends wherein the first branched polyester has a molecular weight of 17000 Daltons and the second branched polyester has a molecular weight of 350,000 Daltons; blends wherein the first branched polyester has a molecular weight of 16000 Daltons and the second branched polyester has a molecular weight of 400,000 Daltons; blends wherein the first branched polyester has a molecular weight of 15000 Daltons and the second branched polyester has a molecular weight of 450,000 Daltons; and blends wherein the first branched polyester has a molecular weight of 10000 Daltons and the second branched polyester has a molecular weight of 500,000 Daltons.

In another aspect, the blends of polyesters disclosed herein comprise the branched polyesters, such as star polyesters, blended together with a linear polyester, such as a linear polyester of a polyol. For example, a low molecular weight branched polyester of a polyol can be blended together with a higher molecular weight linear polyester. Alternatively, a high molecular weight branched polyester of a polyol can be blended together with a linear polyester. The particular blend and choice of branched polyesters determined by the amount of reduction in burst desired, whether the formulation is designed as a liquid or solid, and, when solid, the shape of the blended polyester.

The disclosed blends can comprise at least one disclosed branched polyester, together with another disclosed branched polyester and/or a linear polyester, wherein the branched and/or linear polyester comprises one or more residues of lactide, glycolide, caprolactone, dioxanone, or trimethylene carbonate, such as poly(lactide-co-glycolide).

When the blends comprise a linear polyester, the polyester can have a molecular weight of up to 500,000 Daltons, for example 300,000 Daltons or higher molecular weights blended together with any molecular weight branched polyester (including but not limited to branched polyesters between 1000 and 500,000 Daltons, e.g., a branched polyester with a molecular weight from 20,000 to 200,000 Daltons). For example, in one aspect the linear polyester can have a molecular weight from 1,000 to 500,000; 1,000, to 300,000; 1,000 to 100,000; 1,000 to 50,000; 1,000 to 25,000; 1,000 to 20,000; 1,000 to 10,000; 10,000 to 50,000; 10,000 to 300,000; 10,000 to 100,000; 10,000 to 50,000; 50,000 to 500,000; 50,000 to 300000; 50,000 to 200,000; 50,000 to 100,000; 100,000 to 500,000; 100,000 to 300,000; 100,000 to 200,000; 200,000 to 500,000; 200,000 to 300,000 or 300,000 to 500,000 Daltons and the branched polyester can have a molecular weight form 1,000 to 500,000; 1,000, to 300,000; 1,000 to 100,000; 1,000 to 50,000; 1,000 to 25,000; 1,000 to 20,000; 1,000 to 10,000; 10,000 to 50,000; 10,000 to 300,000; 10,000 to 100,000; 10,000 to 50,000; 50,000 to 500,000; 50,000 to 300000; 50,000 to 200,000; 50,000 to 100,000; 100,000 to 500,000; 100,000 to 300,000; 100,000 to 200,000; 200,000 to 500,000; 200,000 to 300,000 or 300,000 to 500,000 Daltons. For example, a blend of a linear polyester having a molecular weight of 1000 Daltons and a branched polyester having a molecular weight of 50,000 Daltons; a blend of a linear polyester having a molecular weight of 10,000 Daltons and a branched polyester having a molecular weight of 100,000 Daltons; a blend of a linear polyester having a molecular weight of 10,000 Daltons and a branched polyester having a molecular weight of 50,000 Daltons; a blend of a linear polyester having a molecular weight of 10,000 Daltons and a branched polyester having a molecular weight of 75,000 Daltons; a blend of a linear polyester having a molecular weight of 100,00 Daltons and a branched polyester having a molecular weight of 10,000 Daltons; or a blend of a linear polyester having a molecular weight of 20,000 Daltons and a branched polyester having a molecular weight of 500,000 Daltons.

In certain formulations, it can be useful to have a disclosed branched polyester having a molecular weight of less than 20,000 Daltons together with a linear polyester, such as poly(lactide-co-glycolide), having a molecular weight greater than 20,000 Daltons, for example to provide desirable viscosity or release properties of a formulation. Thus, disclosed herein in one aspect are blends of polyesters comprising a low molecular weight branched polyester of a polyol can be blended together with a higher molecular weight linear polyester. For example, disclosed herein are blends comprising a star-type branched polyester of a polyol together with a linear polyester, wherein the branched polyester has a molecular weight less than 19990, 19980, 19970, 19960, 19950, 19900, 19800, 19700, 19600, 19500, 19400, 19300, 19200, 19000, 18500, 18000, 17500, 17000, 16500, 16000, 15500, 15000, 14500, 14000, 13500, 13000, 12500, 12000, 11500, 11000, 10500, 10000, 9000, 8000, 7000, 6000, 5000, 4000, 3000, 2000, or 1000 Daltons (for example, a branched polyester with a molecular weight from 1000 to 20000; from 1000 to 19900; from 1000 to 19500, from 1000 to 19000; from 1000 to 18000; from 1000 to 17000; from 1000 to 16000; from 1000 to 15000; from 1000 to 10000; from 1000 to 5000; from 5000 to 20000; from 5000 to 19900; from 5000 to 195000; from 5000 to 19000; from 5000 to 18000; from 5000 to 17000; from 5000 to 16000; from 5000 to 15000; from 5000 to 10000; from 10000 to 20000; from 10000 to 19900; from 10000 to 19500; from 10000 to 19000; from 10000 to 18000; from 10000 to 17000; from 10000 to 16000; from 10000 to 15000; from 15000 to 20000; from 15000 to 19900; from 15000 to 19500; from 15000 to 19000; from 15000 to 18000; from 15000 to 17000; or 15000 to 16000 Daltons) and the linear polyester has a molecular weight from 1,000 to 500,000; 1,000, to 300,000; 1,000 to 100,000; 1,000 to 50,000; 1,000 to 25,000; 1,000 to 20,000; 1,000 to 10,000; 10,000 to 50,000; 10,000 to 300,000; 10,000 to 100,000; 10,000 to 50,000; 50,000 to 500,000; 50,000 to 300000; 50,000 to 200,000; 50,000 to 100,000; 100,000 to 500,000; 100,000 to 300,000; 100,000 to 200,000; 200,000 to 500,000; 200,000 to 300,000 or 300,000 to 500,000 Daltons. For example, disclosed herein are linear polyester blends comprising a linear polyester of a polyol disclosed herein having a molecular weight of 1000 Daltons and a branched polyester having a molecular weight of 19000 Daltons; blends comprising a linear polyester of a polyol disclosed herein having a molecular weight of 1000 Daltons and a branched polyester having a molecular weight of 210000 Daltons. Also disclosed herein are blends comprising a linear polyester having a molecular weight of 50000 Daltons and a branched polyester having a molecular weight of 5000 Daltons and blends comprising a linear polyester having a molecular weight of 50000 Daltons and a branched polyester having a molecular weight of 210000 Daltons.

Also disclosed are blends of branched polyesters and linear polyesters, wherein a high molecular weight branched polyester (i.e., Mw greater than 200,000 Daltons) is blended with a linear polyester, such as a star-type branched polyester of a polyol blended with a linear polyester of a polyol, wherein the branched polyester has a molecular weight 200100, 200200, 200300, 200400, 200500, 200600, 200700, 200800, 200900, 201000, 202000, 203000, 204000, 205000, 210000, 215000, 220000, 225000, 230000, 235000, 240000, 245000, 250000, 275000, 300000, 350000, 400000, 450,000, or 500,000 Daltons (for example, a branched polyester with a molecular weight from 200,000 to 500,000; from 200,000 to 450,000; from 200,000 to 400,000; from 200,000 to 350,000; from 200,000 to 300,000; from 200,000 to 250,000; from 210,000 to 500,000; from 210,000 to 450,000; from 210,000 to 400,000; from 210,000 to 350,000; from 210,000 to 300,000; from 210,000 to 250,000; from 220,000 to 500,000; from 220,000 to 450,000; from 220,000 to 400,000; from 220,000 to 350,000; from 220,000 to 300,000; from 220,000 to 250,000; from 230,000 to 500,000; from 230,000 to 450,000; from 230,000 to 400,000; from 230,000 to 350,000; from 230,000 to 300,000; from 230,000 to 250,000; from 240,000 to 500,000; from 240,000 to 450,000; from 240,000 to 400,000; from 240,000 to 350,000; from 240,000 to 300,000; from 240,000 to 250,000; from 250,000 to 500,000; from 250,000 to 450,000; from 250,000 to 400,000; from 250,000 to 350,000; from 250,000 to 300,000; from 300,000 to 500,000; from 300,000 to 450,000; from 300,000 to 400,000; from 300,000 to 350,000; from 350,000 to 500,000; from 350,000 to 450,000; from 350,000 to 400,000; from 400,000 to 500,000; 400,000 to 450,000; or from 450,000 to 500,000 Daltons) and the linear polyester has a molecular weight from 1,000 to 500,000; 1,000, to 300,000; 1,000 to 100,000; 1,000 to 50,000; 1,000 to 25,000; 1,000 to 20,000; 1,000 to 10,000; 10,000 to 50,000; 10,000 to 300,000; 10,000 to 100,000; 10,000 to 50,000; 50,000 to 500,000; 50,000 to 300000; 50,000 to 200,000; 50,000 to 100,000; 100,000 to 500,000; 100,000 to 300,000; 100,000 to 200,000; 200,000 to 500,000; 200,000 to 300,000 or 300,000 to 500,000

It is understood and herein contemplated that the blended polyesters can comprise any weight ratio of polyesters to make the disclosed blend. For example, a blend of branched polyesters can comprise 50% weight low molecular weight branched polyester (i.e., a molecular weight less than 20,000 Daltons) and 50% weight high molecular weight polyester (i.e., a molecular weight greater than 200,000 Daltons). Thus, for example, disclosed herein are blends of branched polyesters comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 51, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% weight of a low molecular weight branched polyester and 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1% weight high molecular weight branched polyester.

Similarly, a blend of polyesters comprising a branched polyester and a linear polyester can comprise any percent weight ratio of branched polyester to linear polyester. For example, disclosed herein are linear and branched polyester blends comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 51, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% weight of a branched polyester and 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1% weight of a linear polyester. In one aspect, specifically disclosed are blends comprising comprising 80-99.9% weight linear polyester and 0.1-20% weight branched polyester. For example, a linear polyester of a polyol blended with a branched polyester of a polyol wherein the percentage weight of the linear polyester to the branched polyester is 90% linear polyester and 10% branched polyester.

The specific polymer used as the branched (e.g., comb, brushed graft polymer, or star) or linear polymer can be selected so as to degrade within a desired time interval, once present in a subject, or a biological medium. In some aspects, the time interval can be from about less than one day to about 1 month. Longer time intervals can extend to 6 months, including for example, polymers that degrade from about ≧0 to about 6 months, or from about 1 to about 6 months. In other aspects, the polymer can degrade in longer time intervals, up to 2 years or longer, including, for example, from about ≧0 to about 2 years, or from about 1 month to about 2 years.

The desired bioactive agent release mechanism can influence the selection of the linear and/or branched polymer. A biocompatible polyester, for example, can be selected so as to release or allow the release of a bioactive agent therefrom at a desired lapsed time after the formulation has been administered to a subject. For example, the polyester can be selected to release or allow the release of the bioactive agent prior to the bioactive agent beginning to diminish its activity, as the bioactive agent begins to diminish in activity, when the bioactive agent is partially diminished in activity, for example at least 25%, at least 50% or at least 75% diminished, when the bioactive agent is substantially diminished in activity, or when the bioactive agent is completely gone or no longer has activity.

Examples of suitable polymers that can form branched polyesters with the polyol (or can be used as a linear polymer in the blend) include without limitation polyhydroxyalkanoates, polyhydroxybutyrates, polydioxanones, polyhydroxyvalerates, polyanhydrides, polyorthoesters, polyphosphazenes, polyphosphates, polyphosphoesters, polydioxanones, polyphosphoesters, polyphosphates, polyphosphonates, polyphosphates, polyhydroxyalkanoates, polycarbonates, polyalkylcarbonates, polyorthocarbonates, polyesteramides, polyamides, polyamines, polypeptides, polyurethanes, polyalkylene alkylates, polyalkylene oxalates, polyalkylene succinates, polyhydroxy fatty acids, polyacetals, polycyanoacrylates, polyketals, polyetheresters, polyethers, polyalkylene glycols, polyalkylene oxides, polyethylene glycols, polyethylene oxides, polypeptides, polysaccharides, or polyvinyl pyrrolidones. Other non-biodegradable but durable polymers include without limitation ethylene-vinyl acetate co-polymer, polytetrafluoroethylene, polypropylene, polyethylene, and the like. Likewise, other suitable non-biodegradable polymers include without limitation silicones and polyurethanes.

Further specific examples of suitable polyesters, which can be used in the branched polyester and/or linear polyester, include one or more of a poly(lactide), a poly(glycolide), a poly(lactide-co-glycolide), a poly(caprolactone), a polydioxanone, a poly(trimethylene carbonate), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof

When a block copolymer is desired, examples of useful biodegradable polymers are those that comprise one or more blocks of hydrophilic or water soluble polymers, including, but not limited to, polyethylene glycol, (PEG), or polyvinyl pyrrolidone (PVP), in combination with one or more blocks another biocompatible or biodegradable polymer that comprises lactide, glycolide, caprolactone, dioxanone, or trimethylene carbonate, or a combination thereof.

In one aspect, useful biocompatible polyesters are those that comprise one or more residues of lactic acid, glycolic acid, lactide, glycolide, caprolactone, dioxanone, trimethylene carbonate, hydroxybutyrate, hydroxyvalerates, dioxanones, polyethylene glycol (PEG), polyethylene oxide, or a combination thereof In a still further aspect, useful biocompatible polymers are those that comprise one or more residues of lactide, glycolide, caprolactone, dioxanone, or trimethylene carbonate, or a combination thereof Thus, specifically contemplated are homopolymers, copolymers comprising two or more homopolymers, or terpolymers of lactide, glycolide, caprolactone, dioxanone, and/or trimethylene carbonate, combinations, or bends thereof

Lactide-based polymers can comprise any lactide residue, including all racemic and stereospecific forms of lactide, including, but not limited to, L-lactide, D-lactide, and D,L-lactide, or a mixture thereof Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL-polymers.

In a particular aspect, when the branched or linear polyester is poly(lactide-co-glycolide), or a mixture of poly(lactide) and poly(glycolide), the amount of lactide and glycolide in the polymer can vary. In a further aspect, the polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the polymer can be poly(lactide), 95:5 poly(lactide-co-glycolide) 85:15 poly(lactide-co-glycolide), 75:25 poly(lactide-co-glycolide), 65:35 poly(lactide-co- glycolide), or 50:50 poly(lactide-co-glycolide), where the ratios are mole ratios.

In another aspect, the polymer can be a poly(caprolactone) or a poly(lactide-co-caprolactone). In one aspect, the polymer can be a poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co-caprolactone), or 50:50 poly(lactide-co-caprolactone), where the ratios are mole ratios.

In still a further aspect, when the branched or linear polyester is a terpolymer of one or more of poly(lactide), poly(glycolide), poly(caprolactone), poly(dioxanone), and/or poly(trimethylene carbonate) or mixture of any combination thereof, the amount of the combined homopolymers is 100 mole % and the ratio of the components therein can be any mole ratio of the particular combinations totaling to 100 mole %. For example, 33.33:33.33:33.33; 50:25:25; 60:20:20; 70:15:15; 80:10:10; 90:5:5; 50:30: 20; 60;30;10; and 75:20:5.

A specific example of a branched polyester is a poly(lactide-co-glycolide) ester of a polyol, such as glucose. The poly(lactide-co-glycolide), as discussed above, is formed by initiating polymerization at each hydroxyl group of a polyol initiator (e.g., glucose), and is therefore branched. Such a polyester can be present in a blend with another similar polyester, as discussed above, and/or with another linear polyester, such as linear poly(lactide-co-glycolide).

The disclosed polyol polyesters are particularly suitable to incorporate bioactive agents and produce extended release effects of the active agents in a subject, such as a human. Accordingly, the polyol polyesters and/or blends can be used to prepare microparticles containing an encapsulated bioactive active agent. The polyol polyesters can also be used in other formulations as well as implant devices. Thus, in one aspect, disclosed herein are microparticles, implants, and other formulations comprising the disclosed polymers and blends.

The release characteristics of the formulations, such as microparticles, can be modulated by changing the composition and/or molecular weight of the star and/or linear polymer. Specifically, the balance of hydrophobic and hydrophilic factors can be regulated by changing the ratio of monomers, e.g., changing the lactide to glycolide ratio, and by having different end groups, e.g., ester end groups and acid end groups.

Microparticles formed from the disclosed polyol polyesters can be made according to known methods, such as emulsion methods. For example, emulsions can be made by passing two phases of the emulsion through a column such as a packed bed column, for example, a FormEZE column which is packed with acid washed glass beads and contains an inner phase of water. The emulsion forms as the two fluids, or phases (typically oil and water), are flowing through the gaps inside the packing. As the two phases are flowing through the bed of solids, they cross each other's path repeatedly, and the continuous phase (usually the water) is dividing the discontinuous phase (usually the oil) into droplets, thus creating an emulsion. The resulting oil droplets of the emulsion can contain drug mixed in a solution of the disclosed polyol polyesters. The polymer solvent is subsequently removed from the oil droplets, e.g., by extraction or evaporation, causing the polymer to precipitate, trap the drug and form microparticles.

Implant devices and other formulations are likewise prepared according to known methods, such as melt or wet spinning extrusion methods. The bioactive agent can be present in a formulation in any desired weight percent, for example from 0.5% up to 95% by weight.

Virtually any type of implantable formulation comprising the polyesters disclosed above can be prepared. The implant can comprise any shape, such as a rod, a fiber, a cylinder, a bead, a ribbon, a disc, a wafer, a film, a free-formed shaped solid, or a variety of other shaped solids. The implant can have any regular or irregular shape and can have any cross section like circular, rectangular, triangular, oval, and the like. The injectable or implantable formulation can also comprise drug in a liquid polymer of the disclosed polyol polyester. In one aspect, the implant comprises a cylindrical disk-shape.

Microparticles made with linear polyesters alone can burst drug. Thus, herein disclosed are microparticles comprising a matrix formed from a branched polyester and a linear polyester and a bioactive agent encapsulated therein to reduce the burst of the bioactive agent. For example disclosed herein are microparticles comprising a matrix formed from a linear polyester and a branched polyester and a bioactive agent encapsulated therein to reduce the burst of the bioactive agent, wherein the branched polyester comprises one or more residues of lactide, glycolide, caprolactone, dioxanone, or trimethylene carbonate. In a further aspect, disclosed herein are microparticles wherein the polyol is glucose. The branched polyesters of a polyol in the disclosed microparticles can have a molecular weight of less than 20,000 Daltons, or, alternatively, greater than 200,000 Daltons. The linear polyester can comprise one or more residues of lactide, glycolide, caprolactone, dioxanone, or trimethylene carbonate.

Examples of bioactive agents that can be incorporated into the formulations include without limitation small molecules, peptides, proteins such as hormones, enzymes, antibodies, receptor-binding proteins, antibody fragments, antibody conjugates, nucleic acids such as aptamers, mRNA, siRNA, microRNA, DNA, RNA, antisense nucleic acid or the like, antisense nucleic acid analogs or the like, VEGF inhibitors, macrocyclic lactones, dopamine agonists, dopamine antagonists, low-molecular-weight compounds, high-molecular-weight compounds, or conjugated bioactive agents.

Other bioactive agents can include anabolic agents, antacids, anti-asthmatic agents, anti-cholesterolemic and anti-lipid agents, anti-coagulants, anti-convulsants, anti-diarrheals, anti-emetics, anti-infective agents including antibacterial and antimicrobial agents, anti-inflammatory agents, anti-manic agents, antimetabolite agents, anti-nauseants, anti-neoplastic agents, anti-obesity agents, antipsychotics, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, immunomodulating agents, ion exchange resins, laxatives, mineral supplements, mucolytic agents, neuromuscular drugs, peripheral vasodilators, psychotropics, sedatives, stimulants, thyroid and anti-thyroid agents, tissue growth agents, uterine relaxants, vitamins, or antigenic materials.

Still other bioactive agents include androgen inhibitors, polysaccharides, growth factors, hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Representative drugs that can be used as bioactive agents include, but are not limited to, peptide drugs, protein drugs, therapeutic antibodies, anticalins, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, drugs for metabolic diseases, anti-obesity drugs, estrogens, progestational agents, humoral agents, prostaglandins, analgesics, antispasmodics, antimalarials, antihistamines, cardioactive agents, anti-inflammatory agents, nonsteroidal anti-inflammatory agents, antiparkinsonian agents, antihypertensive agents, β-adrenergic blocking agents, nutritional agents, anti-TNF agents and the benzophenanthridine alkaloids. The agent can further be a substance capable of acting as a stimulant, sedative, hypnotic, analgesic, anticonvulsant, and the like.

Other bioactive agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, and the like; anticoagulants such as heparin, warfarin, and the like; anticonvulsants such as phenytoin sodium, diazepam, and the like; antidepressants such as isocarboxazid, amoxapine, and the like;antihistamines such as diphenhydramine HCl, chlorpheniramine maleate, and the like; antipsychotics such as clozapine, haloperidol, carbamazepine, gabapentin, topimarate, bupropion, sertraline, alprazolam, buspirone, risperidone, aripiprazole, olanzapine, quetiapine, ziprasidone, iloperidone, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B12, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, octreotide, growth hormone, glucagon-like peptides, PYY peptides, growth releasing factor, angiotensin, FSH, EGF, bone morphogenic protein (BMP), erythopoeitin (EPO), interferon, interleukin, collagen, fibrinogen, insulin, Factor VIII, Factor IX, ENBREL®, RITUXAN®, HERCEPTIN®, alpha-glucosidase, CERAZYME/CEREDOSE®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The bioactive agent can also be an immunomodulator, including, for example, cytokines, interleukins, interferon, colony stimulating factor, tumor necrosis factor, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcuc neoformans, Histoplasma capsulatum, Candida albicans, Candida tropicalis, Nocardia asteroids, Rickettsia ricketsii, Rickettsia typhi, Mycoplasma pneumoniae, Chlamyda psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the bioactive agent comprises an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Tinidazole, Ropinerole, Ivermectin, Moxidectin, Afamelanotide, Cilengitide, or a combination thereof. In one aspect, the bioactive agent can be a combination of Rifampicin (Rifampin in U.S.) and Minocycline.

Specific examples of formulations comprise a microparticle or implant device prepared from or comprising a branched or star poly(lactide-co-glycolide) ester of glucose and also comprising octreotide or illiperidone as the bioactive agent. Such formulations can also be prepared from blends, such as a blend of a first low molecular weight branched poly(lactide-co-glycolide) ester of glucose together with a second branched high molecular weight poly(lactide-co-glycolide), as discussed above. Additionally, a low molecular weight branched poly(lactide-co-glycolide) ester of glucose can be formulated with a high molecular weight linear polyester, such as poly(lactide-co-glycolide), as discussed above. Any molecular weight branched polyol polyester can also be blended with any molecular weight linear polymer.

The disclosed formulations can be used to delivery the bioactive agent to a subject, under a desired release profile. The subject can be a vertebrate, such as a mammal, a fish, a bird, a reptile, or an amphibian. Examples include a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. Dosages and particular formulations can be determined by one of skill in the pharmaceutical arts and will vary widely depending on the indication being treated.

As noted above, the branched polyesters and polyester blends disclosed herein are useful in decreasing burst release of a bioactive agent from a formulation, such as a microparticle, when present in a biological environment, such as within a subject. Thus, in one aspect, disclosed herein are methods for making a formulation that exhibits reduced burst of a bioactive agent. Such methods comprise blending a branched polyester, such as a star-type branched polyester of a polyol, with a second branched polyester or a linear polyester of a polyol. Thus, disclosed herein are methods for making a formulation that exhibits reduced burst of a bioactive agent, comprising blending a first branched polyester of a polyol together with a second branched polyester of a polyol; and formulating the blend with a bioactive agent. In another aspect, disclosed herein are methods for making a formulation that exhibits reduced burst of a bioactive agent, comprising blending a branched polyester of a polyol together with a linear polyester; and formulating the blend with a bioactive agent. The disclosed methods can utilize any of the branched polyesters and polyester blends disclosed herein, such as a star-type branched polyester or a blend of a star-type branched polyester and a linear polyester. In a further aspect, the branched polyester of the polyol used in the disclosed methods has a molecular weight of less than 20,000 Daltons, or, alternatively, greater than 200,000 Daltons. For example, where two branched polyesters are employed the first polyester of the polyol can have a molecular weight of less than 20,000 Daltons and/or the second branched polyester of the polyol can have a molecular weight of greater than 200,000 Daltons. In still a further aspect, disclosed are methods of decreasing burst release by making a branched polyester/linear polyester blend, wherein the linear polyester has a molecular weight of up to 300,000 Daltons.

In another aspect, disclosed herein are methods for modulating the burst release of a bioactive agent from a formulation such as a microparticle comprising blending a branched polyester of a polyol such as a start polyester of a polyol with a linear polymer.

In a first aspect, disclosed herien are branched polyesters comprising branched polyesters of a polyol having a molecular weight of less than 20,000 Daltons, or, alternatively, greater than 200,000 Daltons; wherein the polyester comprises one or more residues of lactide, glycolide, caprolactone, dioxanone, or trimethylene carbonate.

Also disclosed are branched polyesters of the first aspect, wherein the polyester is a star polyester.

Also disclosed are branched polyesters of the first aspect, which comprises a poly(lactide-co-glycolide) ester of the polyol.

Also disclosed are the branched polyesters of any preceding aspect, wherein the polyol is glucose.

In a fifth aspect, disclosed herein are blends comprising the branched polyester of any preceding aspect together with a linear polyester having a molecular weight of up to 500,000 Daltons.

Also disclosed are the blends of the fifth aspect, wherein the linear polyester comprises one or more residues of lactide, glycolide, caprolactone, dioxanone, or trimethylene carbonate.

Also disclosed are the blends of aspect 5 or 6, wherein the linear polyester comprises poly(lactide-co-glycolide).

In an eighth aspect, disclosed herein are blends comprising a first branched polyester of a polyol having a molecular weight of less than 20,000 Daltons; and a second branched polyester of a polyol having a molecular weight greater than 200,000 Daltons; wherein each of the first and second polyesters independently comprises one or more residues of lactide, glycolide, caprolactone, dioxanone, or trimethylene carbonate.

In an ninth aspect, disclosed herein are microparticles comprising a matrix formed from the branched polyester or blend of any preceding aspect and a bioactive agent encapsulated therein.

Also disclosed are the microparticle of the ninth aspect, wherein the bioactive agent comprises a peptide or protein.

Also disclosed are the microparticle of any preceding aspect, wherein the bioactive agent comprises octreotide.

In a twelfth aspect, disclosed herein is a microparticle comprising a matrix formed from a branched polyester and a linear polyester and a bioactive agent encapsulated therein to reduce the burst of the bioactive agent.

Also disclosed are the microparticles of the twelfth aspect, wherein the branched polyester comprises a polyester of a polyol having a molecular weight of less than 20,000 Daltons, or, alternatively, greater than 200,000 Daltons.

Also disclosed are the microparticles of aspects 12 or 13, wherein the branched polyester comprises one or more residues of lactide, glycolide, caprolactone, dioxanone, or trimethylene carbonate

Also disclosed are the microparticles of aspects 13 or 14, wherein the polyol is glucose.

In a sixteenth aspect, disclosed herien are methods for making a formulation that exhibits reduced burst of a bioactive agent, comprising blending a first branched polyester of a polyol together with a second branched polyester of a polyol; and formulating the blend with a bioactive agent.

Also disclosed are the methods of aspect 16, wherein the first branched polyester of the polyol has a molecular weight of less than 20,000 Daltons.

Also disclosed are the methods of aspects 16 or 17, wherein the second branched polyester of the polyol has a molecular weight greater than 200,000 Daltons.

In a nineteenth aspect, disclosed herien are methods for making a formulation that exhibits reduced burst of a bioactive agent, comprising blending a branched polyester of a polyol together with a linear polyester; and formulating the blend with a bioactive agent.

Also disclosed are the methods of aspect 19, wherein the branched polyester of the polyol has a molecular weight of less than 20,000 Daltons, or, alternatively, greater than 200,000 Daltons.

Also disclosed are the methods of aspect 19 or 20, wherein the linear polyester has a molecular weight of up to 300,000 Daltons.

In a twenty-second aspect, disclosed herein are methods for modulating the burst release of a bioactive agent from a formulation such as a microparticle comprising blending a branched polyester of a polyol with a linear polyester.

Various modifications and variations can be made to the compositions and formulations disclosed herein. Other aspects of the compositions and formulations described herein will be apparent from consideration of the specification and practice of the composites, kits, articles, devices, compositions, and methods disclosed herein. It is intended that the specification and examples be considered as exemplary.

What is claimed is:

1. A branched polyester comprising a branched polyester of a polyol having a molecular weight of less than 20,000 Daltons, or, alternatively, greater than 200,000 Daltons; wherein the polyester comprises one or more residues of lactide, glycolide, caprolactone, dioxanone, or trimethylene carbonate.

2. The branched polyester of claim 1, wherein the polyester is a star polyester.

3. The branched polyester of claim 1, which comprises a poly(lactide-co-glycolide) ester of the polyol.

4. The branched polyester of claim 1, wherein the polyol is glucose.

5. A blend comprising the branched polyester of claim 1 together with a linear polyester having a molecular weight of up to 500,000 Daltons.

6. The blend of claim 5, wherein the linear polyester comprises one or more residues of lactide, glycolide, caprolactone, dioxanone, or trimethylene carbonate.

7. The blend of claim 5, wherein the linear polyester comprises poly(lactide-co-glycolide).

8. A blend of branched polyesters of claim 1 comprising a first branched polyester of a polyol having a molecular weight of less than 20,000 Daltons; and a second branched polyester of a polyol having a molecular weight greater than 200,000 Daltons; wherein each of the first and second branched polyesters independently comprises one or more residues of lactide, glycolide, caprolactone, dioxanone, or trimethylene carbonate.

9. A microparticle comprising a matrix formed from the branched polyesters or blend of the branched polyesters of claim 1 and a bioactive agent encapsulated therein.

10. The microparticle of claim 9, wherein the bioactive agent comprises a peptide or protein.

11. The microparticle of claim 9, wherein the bioactive agent comprises octreotide.

12. A microparticle comprising a matrix formed from a branched polyester and a linear polyester and a bioactive agent encapsulated therein to reduce the burst of the bioactive agent.

13. The microparticle of claim 12, wherein the branched polyester comprises a polyester of a polyol having a molecular weight of less than 20,000 Daltons, or, alternatively, greater than 200,000 Daltons.

14. The microparticle of claim 12, wherein the branched polyester comprises one or more residues of lactide, glycolide, caprolactone, dioxanone, or trimethylene carbonate.

15. The microparticle of claim 12, wherein the polyol is glucose.

16. A method for making a formulation that exhibits reduced burst of a bioactive agent, comprising blending a first branched polyester of a polyol together with a second branched polyester of a polyol; and formulating the blend with a bioactive agent.

17. The method of claim 16, wherein the first branched polyester of the polyol has a molecular weight of less than 20,000 Daltons.

18. The method of claim 16, wherein the second branched polyester of the polyol has a molecular weight greater than 200,000 Daltons.

19. A method for making a formulation that exhibits reduced burst of a bioactive agent, comprising blending a branched polyester of a polyol together with a linear polyester; and
formulating the blend with a bioactive agent.

20. The method of claim 19, wherein the branched polyester of the polyol has a molecular weight of less than 20,000 Daltons, or, alternatively, greater than 200,000 Daltons.

21. The method of claim 19, wherein the linear polyester has a molecular weight of up to 300,000 Daltons.

22. A method for modulating the burst release of a bioactive agent from a formulation such as a microparticle comprising blending a branched polyester of a polyol with a linear polyester.
